(19) United States
(12) Patent Application Publication
(10) Pub. No.: US 2025/0360261 A1
(43) Pub. Date: Nov. 27, 2025

(54) MODULAR NEGATIVE PRESSURE WOUND THERAPY SYSTEMS

(71) Applicant: Solventum Intellectual Properties Company, Maplewood, MN (US)

(72) Inventors: Benjamin A. PRATT, Bracknell (GB); Robert HOWARD, Bracknell (GB); James K. SEDDON, Bracknell (GB)

(21) Appl. No.: 18/867,247

(22) PCT Filed: May 4, 2023
(86) PCT No.: PCT/IB2023/054647

§ 371 Date: Nov. 19, 2024

Related U.S. Application Data
(60) Provisional application No. 63/345,246, filed on May 24, 2022.

(51) Int. Cl. A61M 1/00; A61F 13/05

(52) U.S. Cl. CPC A61M 1/966; A61F 13/05; A61M 1/80; A61M 2205/3344; A61M 2205/3584; A61M 2205/8206; A61M 2210/04

(57) ABSTRACT

Systems and methods for negative pressure wound therapy. The system includes a dressing, a therapy device, and a pressure sensing device. The dressing is configured to be positioned at the tissue site. The therapy device is configured to fluidly couple to the dressing. The therapy device includes a pump configured to deliver negative pressure to the dressing, a first wireless communication device, and a controller configured to control the pump. The pressure sensing device is configured to be fluidly coupled to the dressing at a sensing location external to the therapy device and to wirelessly communicate a pressure signal from the sensing location to the first wireless communication device.

### CROSS-REFERENCE TO RELATED APPLICATIONS

This application is a U.S. National Stage Entry of International Application No. PCT/IB2023/054647, filed on May 4, 2023, which claims the benefit of priority to U.S. Provisional Application No. 63/345,246, filed on May 24, 2022, each of which are incorporated herein by reference in their entirety.

### TECHNICAL FIELD

The invention set forth in the appended claims relates generally to tissue treatment systems and more particularly, but without limitation, to modular negative pressure wound therapy systems and methods.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for modular therapy devices in a negative-pressure therapy environment are set forth in the appended claims. Illustrative embodiments are also provided to enable a person skilled in the art to make and use the claimed subject matter.

For example, in some embodiments, a system for treating a tissue site is described. The system can include a dressing, a therapy device, and a pressure sensing device. The dressing can be configured to be positioned at the tissue site. The therapy device can be configured to fluidly couple to the dressing. The therapy device can include a pump configured to deliver negative pressure to the dressing, a first wireless communication device, and a controller configured to control the pump. The pressure sensing device can be configured to be fluidly coupled to the dressing at a sensing location external to the therapy device and to wirelessly communicate a pressure signal from the sensing location to the first wireless communication device.

In some example embodiments, the therapy device can further include a pump pressure sensor and a power source. The pump pressure sensor can be configured to sense a pressure output of the pump and the power source can be configured to provide power to the therapy device.

In some example embodiments, the therapy device can further include a pump housing and a battery pack. The pump housing can be configured to contain the pump and can include at least one housing connection configured to provide communication with the pump through the pump housing. The battery pack can be configured to couple to the pump housing. The battery pack can include a power source configured to provide power to the therapy device and at least one battery pack connection configured to provide communication with the power source. The at least one battery pack connection can be configured to couple to the at least one housing connection when the battery pack is coupled to the pump housing. The at least one pump housing and/or the battery pack can include a user interface.

In some example embodiments, the first wireless communication device can be a Bluetooth device.

In some example embodiments, the pressure sensing device can include a printed circuit board, a therapy sensor, an ambient pressure sensor, and a second wireless communication device. The printed circuit board can include a first side and a second side. The therapy pressure sensor can be configured to sense a pressure at the tissue site. The ambient pressure sensor can be configured to sense an ambient environment pressure external to the therapy device and the dressing. The second wireless communication device can be configured to wirelessly communicate a pressure signal to the first wireless communication device. The pressure signal can include a therapy pressure value corresponding to the pressure at tissue site and an ambient pressure value corresponding to the ambient environment pressure.

In some example embodiments, the therapy pressure sensor and the ambient pressure sensor can include a single pressure sensing assembly configured to generate the therapy pressure value relative to the ambient pressure value.

In some example embodiments, the therapy pressure sensor can be coupled to the first side of the printed circuit board and the ambient pressure sensor can be coupled to the second side of the printed circuit board opposite the first side and isolated from the therapy pressure sensor. In some example embodiments, the pressure sensing device can be coupled to a surface of the dressing opposite the tissue site. The therapy pressure sensor on the first side of the printed circuit board can be configured to face the dressing and to be exposed to the pressure at the tissue site.

In some example embodiments, the pressure sensing device is coupled to a surface of the dressing opposite the tissue site. The pressure sensing device can be configured to be exposed to the pressure at the tissue site. The therapy pressure sensor can be fluidly coupled to the tissue site through the dressing.

In some example embodiments, the pressure sensing device can further include a power supply that can be configured to provide power to the pressure sensing device. The power supply can include a battery coupled to the printed circuit board.

In some example embodiments, the second wireless communication device can be a Bluetooth device.

In some example embodiments, the system can further include a conduit configured to fluidly couple the pump of the therapy device to the dressing. The conduit can be a single lumen conduit that can extend from the pump to the dressing. The system can further include a fluid storage container fluidly coupled inline between the dressing and the pump through one or more portions of the single lumen conduit.

In some example embodiments, the pressure sensing device can be carried by a conduit connection assembly configured to be coupled between the therapy device and the dressing. The conduit connection assembly can include a single lumen conduit connection at a first end and a multi-lumen conduit connection at a second end. The single lumen conduit connection can be configured to fluidly couple to a single lumen conduit between the pump and the first end and the multi-lumen conduit connection can be configured to fluidly couple to a multi-lumen conduit between the second end and the dressing. The multi-lumen conduit can include a reduced-pressure pathway fluidly isolated from a sensing pathway. The sensing pathway can be configured to be in fluid communication between the pressure sensing device and the dressing. The reduced-pressure pathway can be configured to be in fluid communication between the dressing and the single lumen conduit fluidly coupled to the pump.

In some example embodiments, the system can further include a fluid storage container configured to be coupled to an exterior of the therapy device and fluidly coupled between the pump and the dressing. The pressure sensing device can be disposed within the fluid storage container. In some example embodiments, the system can further include a negative pressure pathway configured to fluidly couple the pump to the dressing and a sensing pathway configured to fluidly couple the pressure sensing device to the dressing. The pressure sensing device is not fluidly coupled to the therapy device. In some example embodiments, at least a portion of the negative pressure pathway and the sensing pathway are carried by a multi-lumen conduit that can be configured to be fluidly coupled between the fluid storage container and the dressing. In some example embodiments, the sensing pathway can be fluidly isolated from the negative pressure pathway.

In some example embodiments, the pressure sensing device can include an RFID tag that can be configured to communicatively couple the pressure sensing device to the therapy device when the RFID tag is within a sensing range of the therapy device.

Also described herein is another system for treating a tissue site with negative pressure. In some example embodiments, the system can include a dressing, a therapy device, and a pressure sensing device. The dressing can be configured to be positioned at the tissue site. The therapy device can include a pump and a wireless communication device. The pump can be configured to generate a negative pressure and to provide the negative pressure to the dressing through a negative pressure pathway. The pressure sensing device can be configured to be fluidly coupled to the dressing through a sensing pathway and at a sensing location external to the therapy device. The sensing pathway can be fluidly isolated from the negative pressure pathway and the therapy device. The pressure sensing device can be further configured to wirelessly communicate a pressure signal from the sensing location to the wireless communication device.

Also described herein is a method of treating a tissue site with negative pressure. In some example embodiments, the method can include obtaining a therapy device including a pump, a pump pressure sensor, a first wireless communication device, and a controller. The pump can be configured to deliver negative pressure, the pump pressure sensor can be configured to sense a pressure at the pump, and the controller can be configured to control the pump. The method can further include obtaining a pressure sensing device including a printed circuit board, a therapy pressure sensor, an ambient pressure sensor, and a second wireless communication device. The therapy pressure sensor can be configured to sense a pressure at the tissue site, the ambient pressure sensor can be configured to sense an ambient environment pressure, and the second wireless communication device can be configured to wirelessly communicate a pressure signal to the first wireless communication device. The pressure signal can include a therapy pressure value corresponding to the pressure at the tissue site and an ambient pressure value corresponding to the ambient environment pressure. The method can further include fluidly coupling the pump to a dressing disposed at the tissue site, fluidly coupling the pressure sensing device to the dressing disposed at the tissue site, actuating the pump to deliver negative pressure to the dressing, and monitoring, with the pressure sensing device, the dressing while the pump is delivering negative pressure to the dressing.

In some example embodiments, the method can further include collecting fluids from the tissue site in a canister. The canister can be configured to be coupled in fluid communication between the pump and the dressing.

In some example embodiments, monitoring, with the pressure sensing device, the dressing while the pump is delivering negative pressure to the dressing can include comparing the pressure signal and a pump pressure measured by the pump pressure sensor to detect blockages within the dressing or between the therapy device and the dressing.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2A is a front view of an example therapy device that may be associated with some embodiments of the therapy system of FIG. 1;

FIG. 2B is a top view of the therapy device of FIG. 2A;

FIG. 2C is a side view of the therapy device of FIG. 2A;

FIG. 2D is a front exploded view of the therapy device of FIG. 2A;

FIG. 2E is a perspective exploded view of the therapy device of FIG. 2A;

FIG. 2F is a cut-away front view of the therapy device of FIG. 2A;

FIG. 3 is a perspective partial cut-away view of an example embodiment of the therapy device of FIG. 2A;

FIG. 4A is a top view of an example pressure sensing device that may be associated with some embodiments of the therapy system of FIG. 1;

FIG. 4B is a bottom view of the pressure sensing device of FIG. 4A;

FIG. 4C is a side view of the pressure sensing device of FIG. 4A;

FIG. 4D is a top perspective view of the pressure sensing device of FIG. 4A;

FIG. 4E is a bottom perspective view of the pressure sensing device of FIG. 4A;

FIG. 5A is a perspective view of an example embodiment of the therapy system of FIG. 1;

FIG. 5B is a side cut-away view of the therapy system of FIG. 5A;

FIG. 6 is a perspective view of another example embodiment of the therapy system of FIG. 1;

FIG. 7A is a perspective view of another example embodiment of the therapy system of FIG. 1;

FIG. 7B is a side cut-away view of the therapy system of FIG. 7A;

FIG. 8A is a perspective view of another example embodiment of the therapy system of FIG. 1;

FIG. 8B is a side view of a therapy device and a canister of the therapy system of FIG. 8A; and

FIG. 8C is an exploded view of the therapy device and the canister of FIG. 8B.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

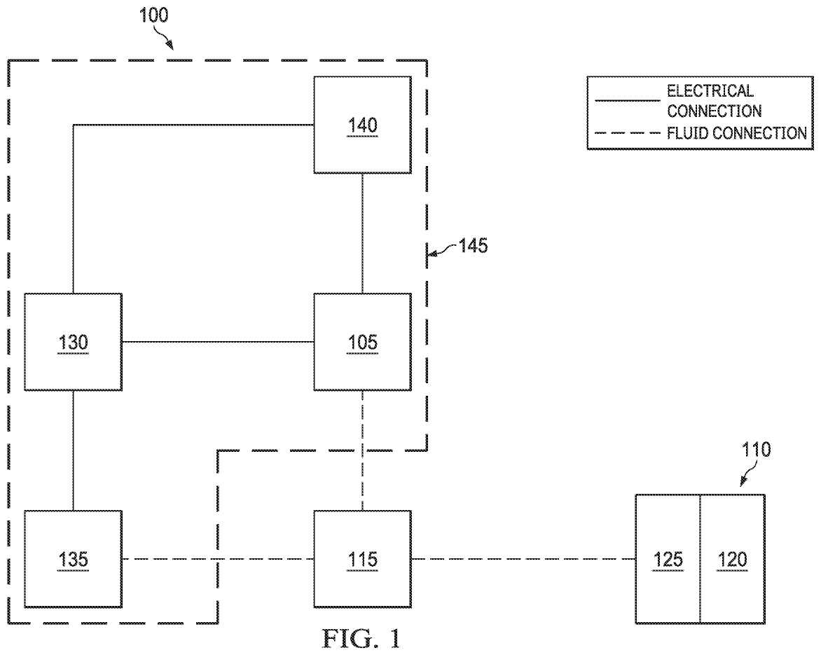

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit or a therapy device 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

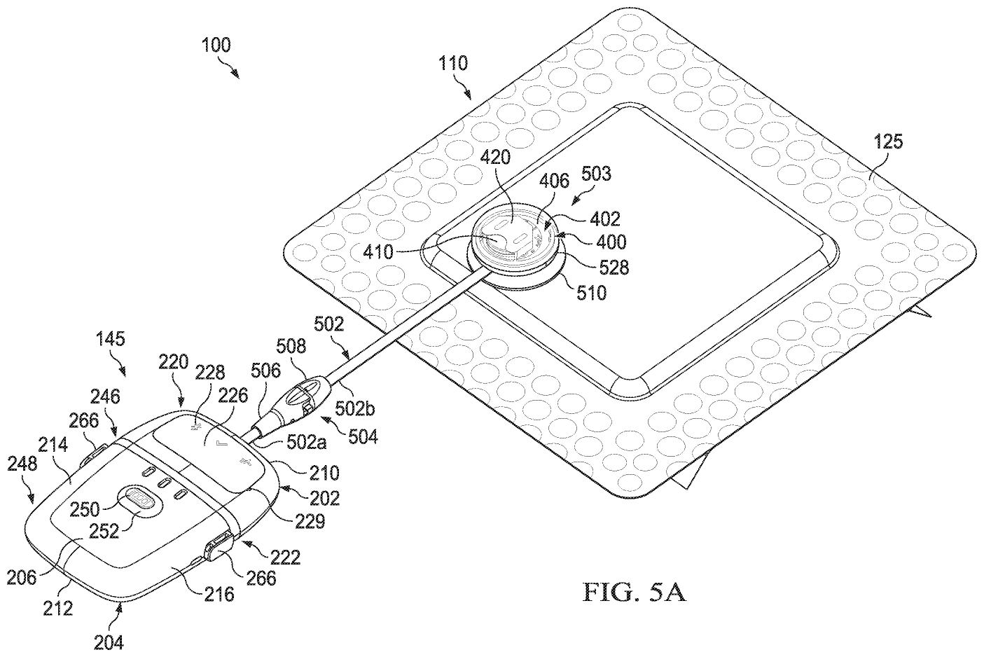

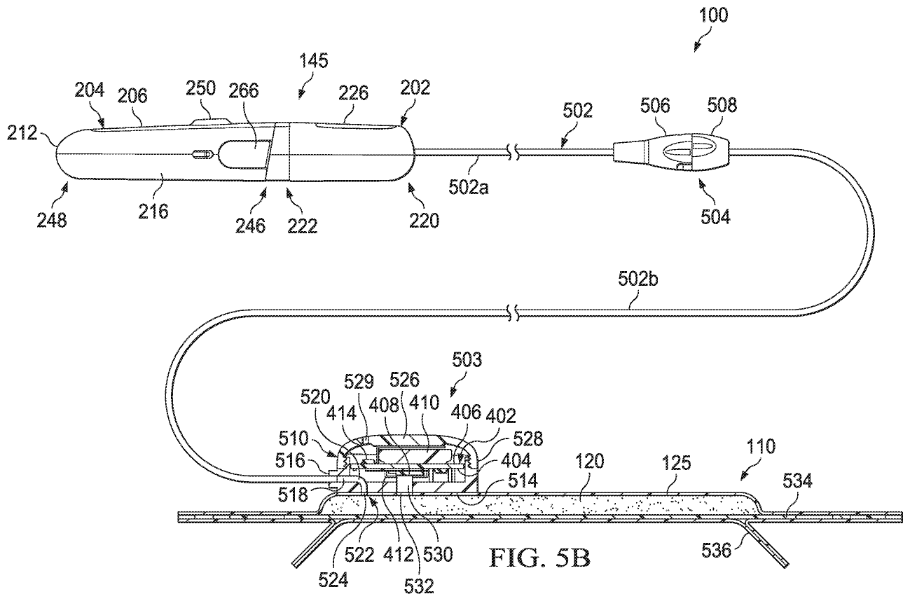

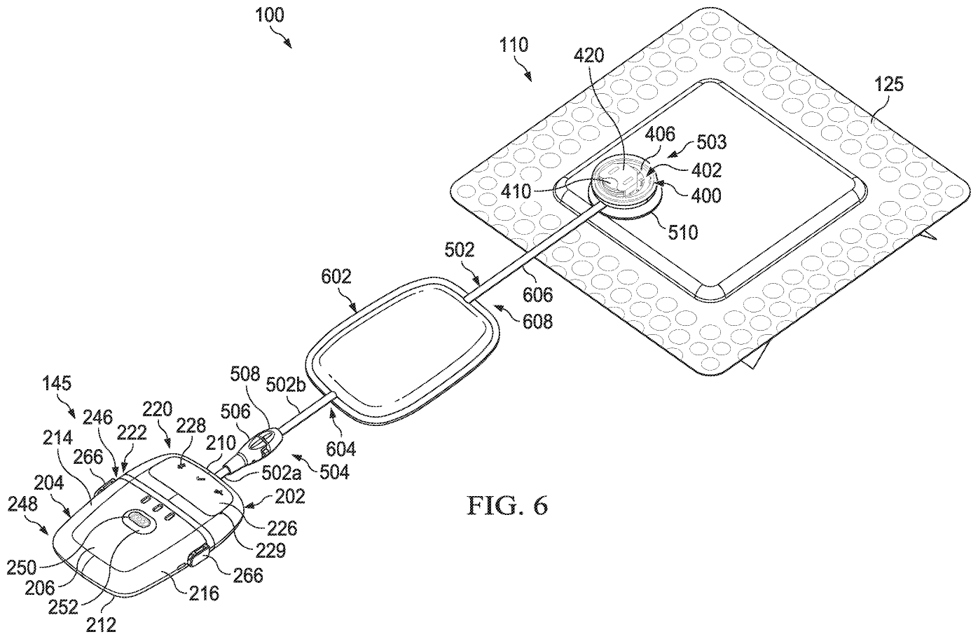

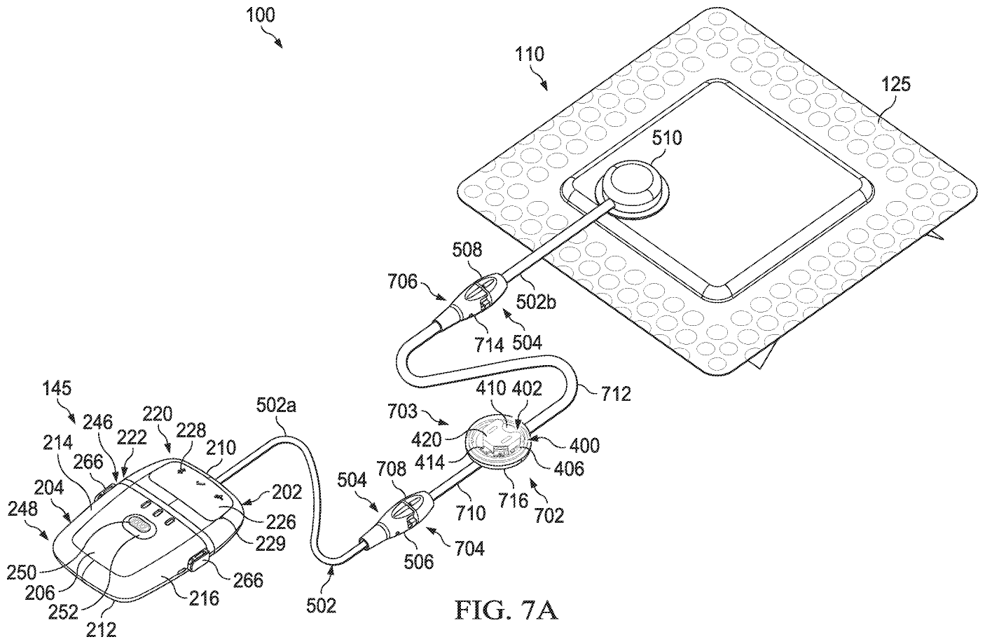

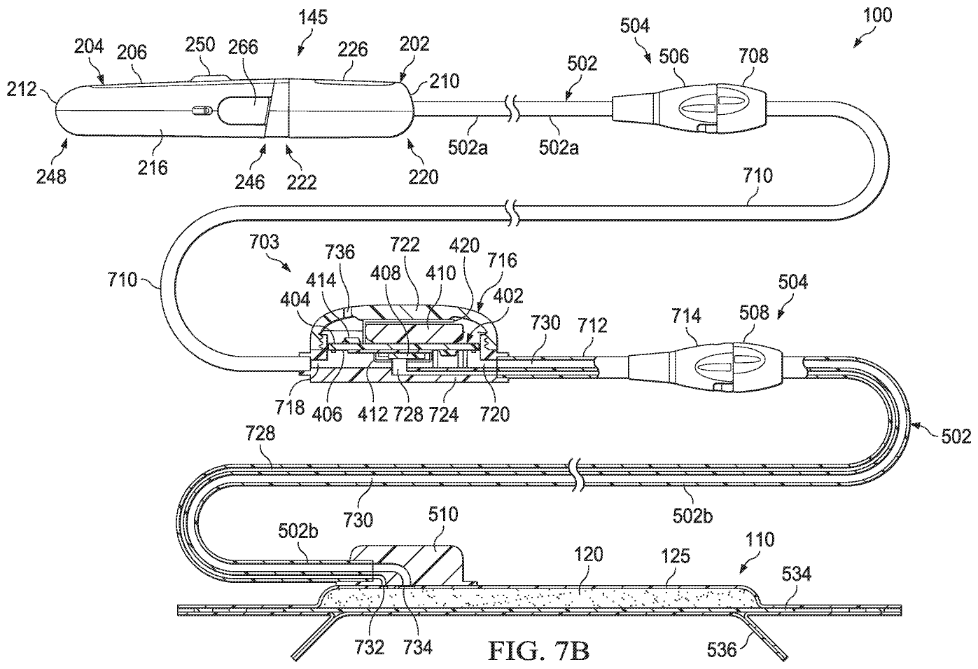

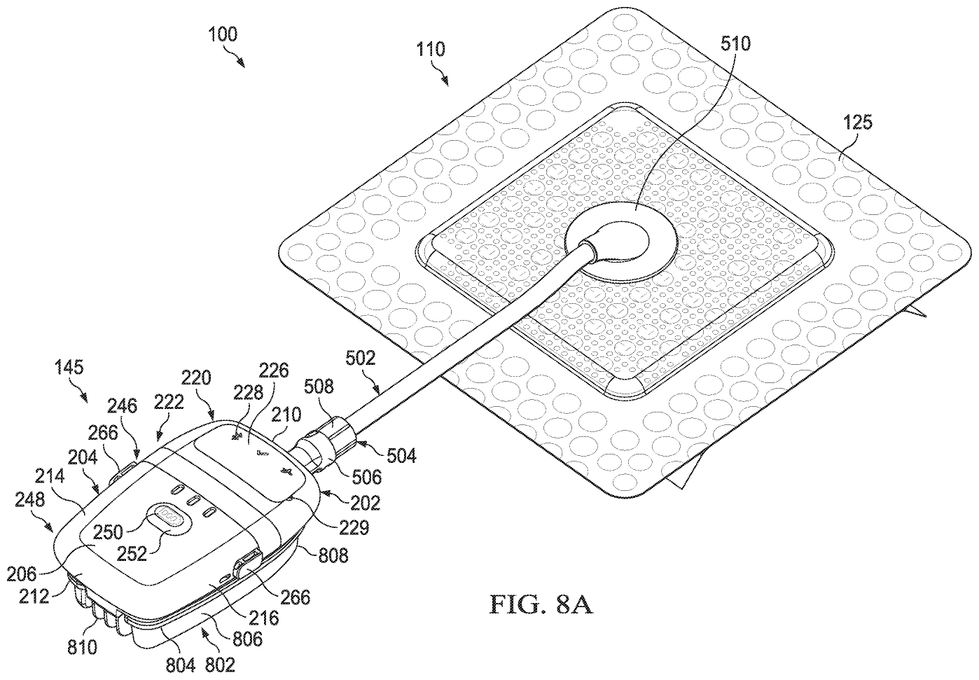

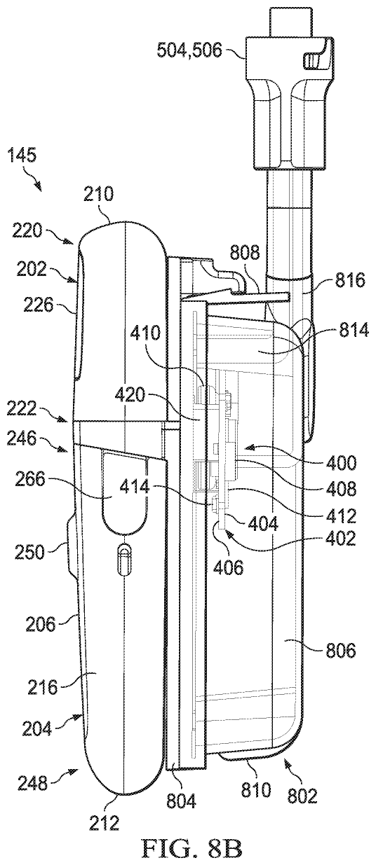

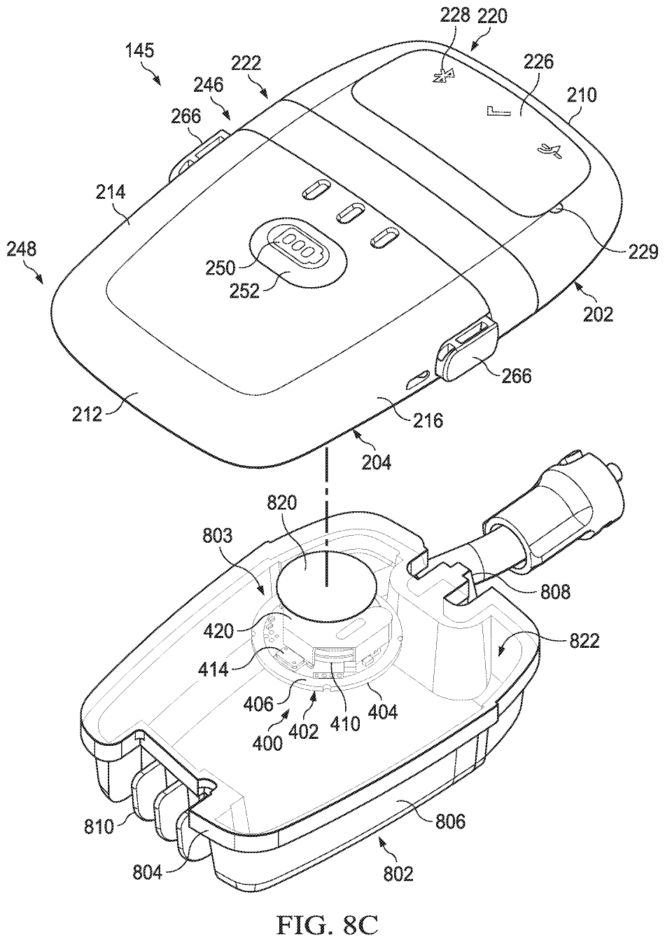

Sensors, such as the first sensor 135 and the second sensor 140, may be any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, which may correspond to a pressure output of the negative-pressure source 105, in some embodiments. Preferably, the signals from the first sensor 135 and the second sensor 140 are suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal. The first sensor 135 and the second sensor 140 are illustrated as optional features of some examples of the therapy device 145 in FIG. 1. However, in some examples, the first sensor 135 and/or the second sensor 140 can be omitted or moved to another portion of the therapy system 100, such as to a location external to the therapy device 145. In one such example, the second sensor 140 can be included as part of the therapy device 145 and the first sensor 135 can be configured as a pressure sensing device that can be fluidly coupled to the dressing 110 at multiple sensing locations external to the therapy device 145 as desired and as described herein. A sensing location can be any location external to the therapy device 145, such as, for example, a sensing location 503 at a dressing as shown in FIGS. 5A and 6, a sensing location 703 at a conduit connection assembly as shown in FIG. 7A, a sensing location 803 associated with a fluid storage container as shown in FIG. 8C, or another external location that is not fluidly coupled to the therapy device 145.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from a tissue site and drawing the fluid toward the source. In some embodiments, the fluid path may be reversed or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and INSPIRER 2301 and INSPIRER 2327 polyurethane films, commercially available from Exopack Advanced Coatings, Wrexham, United Kingdom. In some embodiments, the cover 125 may comprise INSPIRE® 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example.

In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. Similarly, it may be convenient to describe certain features in terms of fluid “inlet” or “outlet” in such a frame of reference. This orientation is generally presumed for purposes of describing various features and components herein. However, the fluid path may also be reversed in some applications, such as by substituting a positive-pressure source for a negative-pressure source, and this descriptive convention should not be construed as limiting.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

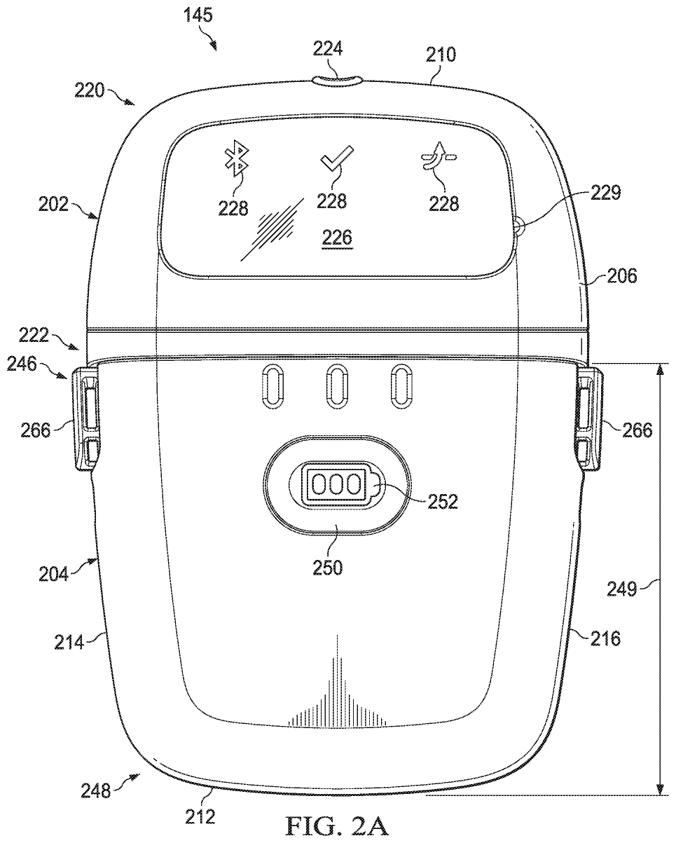

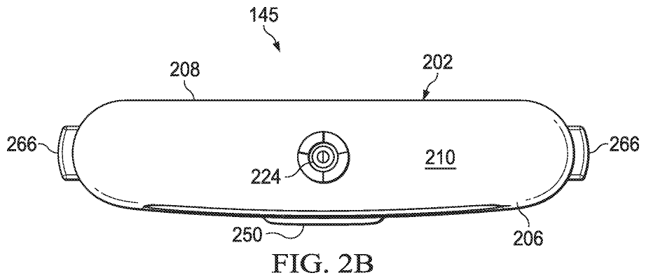

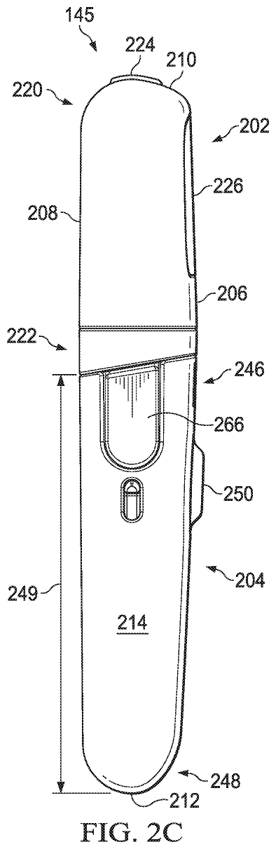

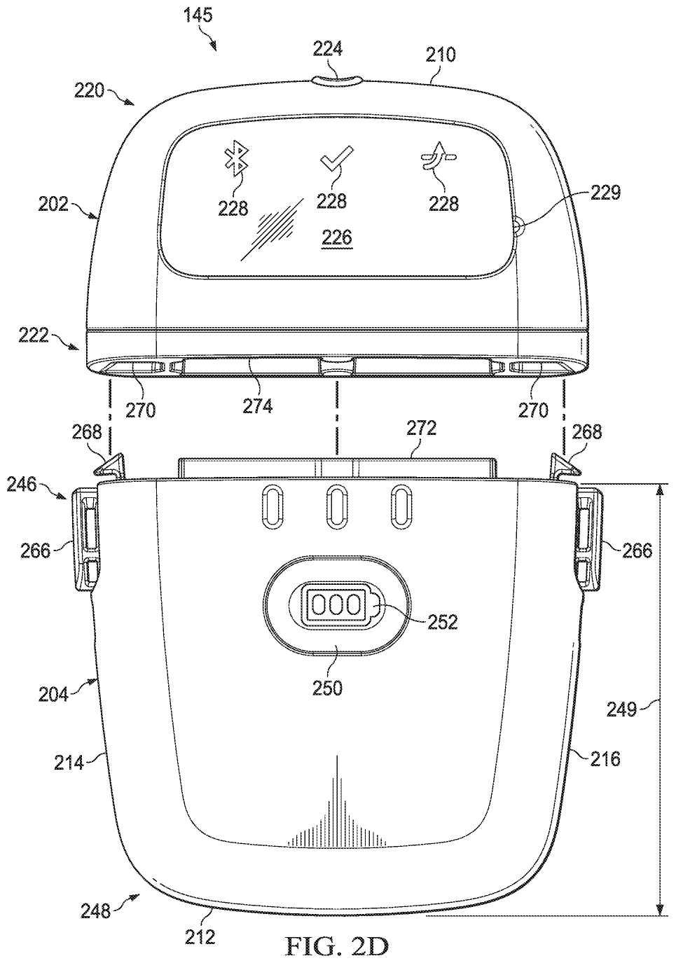

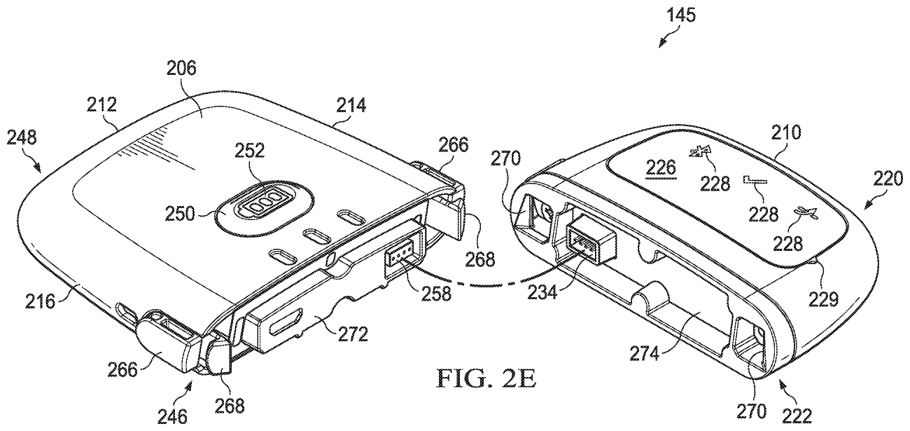

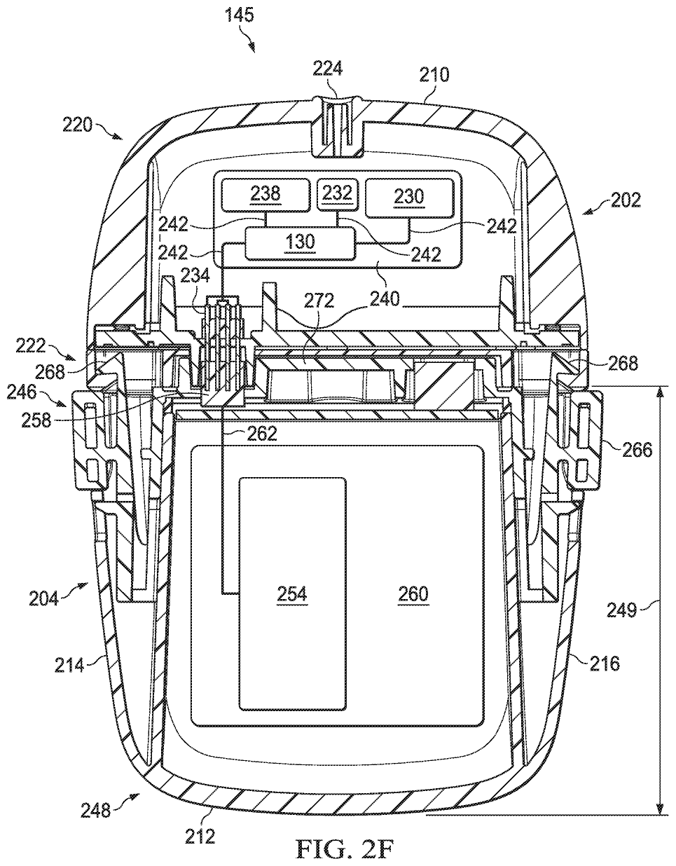

Referring collectively to FIGS. 2A-2F, these figures illustrate multiple views of an example embodiment of the therapy device 145. FIG. 2A is a front view of the therapy device 145. FIG. 2B is a top view of the therapy device 145. FIG. 2C is a side view of the therapy device 145. FIG. 2D is an exploded front view of the therapy device 145. FIG. 2E is an exploded perspective view of the therapy device 145. FIG. 2F is a front cut-away view of the therapy device 145.

The therapy device 145 may include a pump housing 202 and a power module or a battery pack 204. In some embodiments, the therapy device 145 may be substantially rectangular. In other embodiments, the therapy device 145 may be ovular, square, or another shape. The therapy device 145 may have a first surface 206, a second surface 208 opposite the first surface 206, a top surface 210 that may couple the first surface 206 and the second surface 208, a bottom surface 212 opposite the top surface 210, a first side 214 that may couple the top surface 210 and the bottom surface 212 and the first surface 206 and the second surface 208, and a second side 216 opposite the first side 214.

The pump housing 202 may have a first end 220 and a second end 222 opposite the first end 220. The first end 220 of the pump housing 202 may include the top surface 210 of the therapy device 145. There may be a connection point or a dressing connection 224 on the first end 220 of the pump housing 202 in some embodiments. The dressing connection 224 may couple the pump housing 202 to the dressing 110 in some embodiments. For example, a conduit may be coupled between the dressing connection 224 and the dressing 110.

The pump housing 202 may optionally include a user interface 226 in some embodiments. The user interface 226 may be configured to output information related to the therapy device 145. In some embodiments, a user may be able to interact with or control the therapy device 145 via the user interface 226. For example, the user interface 226 may include a touch screen or buttons that allow a user to communicate with the therapy device 145. In some embodiments, the user interface 226 may further include one or more indicators 228. The indicators 228 may be symbols that may change color or appearance when a certain condition is met. For example, one of the indicators 228 may be a Bluetooth® symbol that may light up when the therapy device 145 is communicating with an exterior device via Bluetooth®. In some embodiments, one of the indicators 228 may be a check mark that may light up when the pump housing 202 is coupled to the battery pack 204. The check mark may instead light up when the therapy device 145 is powered on. In some embodiments, one of the indicators 228 may be an airflow symbol that may indicate when a pump is actuated within the therapy device 145. In some embodiments, the one or more indicators 228 may be different symbols that may communicate information about the therapy device 145 to a user.

In some embodiments, the pump housing 202 may further include a port 229. The port 229 may provide communication between an ambient environment and the pump housing 202. In some embodiments the port 229 may be disposed on the first surface 206 of the pump housing 202. In other embodiments, the port 229 may be at a different location of the pump housing 202.

In some embodiments, the pump housing 202 may include the controller 130, a pump 230, a pump pressure sensor 232, and a first communications connector or a housing connection 234. In some embodiments, the pump 230 may be the negative-pressure source 105 and may be configured to deliver a negative pressure to the dressing 110 when the pump 230 is actuated. The pump pressure sensor 232 may be configured to sense a pressure output at the pump 230. The housing connection 234 may be configured to provide communication with the controller 130 and/or the pump 230 from an exterior of the pump housing 202. For example, the housing connection 234 may be configured to communicatively couple with the battery pack 204 of the therapy device 145. In some embodiments, the pump housing 202 may further include a first wireless communication device 238. The first wireless communication device 238 may be a Bluetooth® device in some embodiments. The first wireless communication device 238 may be configured to send and receive signals from an exterior of the pump housing 202.

The controller 130, the pump 230, the pump pressure sensor 232, and the first wireless communication device 238 may each be coupled to a printed circuit board 240. Further, the pump 230, the pump pressure sensor 232, and the first wireless communication device 238 may each be communicatively coupled to the controller 130. Further, the controller 130 may be communicatively coupled with the housing connection 234. In some embodiments, there may be wires 242 coupling the pump 230, the pump pressure sensor 232, and the first wireless communication device 238 to the controller 130. Alternatively or additionally, the components may be wirelessly coupled. The controller 130 may be configured to send signals to and receive signals from each of the pump 230, the pump pressure sensor 232, and the first wireless communication device 238.

The battery pack 204 may have a first end 246 and a second end 248 opposite the first end 246. The battery pack 204 may have a length 249 defined between the first end 246 of the battery pack 204 and the second end 248 of the battery pack 204. The second end 248 of the battery pack 204 may include the bottom surface 212 of the therapy device 145. The battery pack 204 may optionally include a user interface 250 in some embodiments. The user interface 250 may be configured to output information related to the therapy device 145. In some embodiments, a user may be able to interact with or control the therapy device 145 via the user interface 250. For example, the user interface 250 may include a touch screen or buttons that allow a user to communicate with the therapy device 145. In some embodiments, the user interface 250 may be or may include a battery indicator 252. The battery indicator 252 may output a battery level to a user.

The battery pack 204 may include a power source or a battery 254 and a second communications connector or a battery pack connection 258. The battery 254 may be configured to provide power to the therapy device 145. The battery 254 may be rechargeable and/or may be replaceable. In some embodiments, the battery 254 may be coupled to a printed circuit board 260. The battery 254 may be communicatively coupled to the battery pack connection 258. In some embodiments, there may be wires 262 coupling the battery 254 to the battery pack connection 258. In some embodiments, the components may be wirelessly coupled. The battery pack connection 258 may be coupled with the housing connection 234. When the battery pack connection 258 is coupled to the housing connection 234, the battery 254 may be communicatively coupled to the controller 130. In some embodiments, when the battery pack connection 258 is coupled to the housing connection 234 the controller 130 may be configured to send signals to and receive signals from the battery 254.

In some embodiments, the second end 222 of the pump housing 202 may be coupled to the first end 246 of the battery pack 204 with snaps 266. In some embodiments, the therapy device 145 may include one of the snaps 266 on the first side 214 of the therapy device 145 and another of the snaps 266 on the second side 216 of the therapy device 145. The snaps 266 may enable a user to release the pump housing 202 from the battery pack 204. In some embodiments, the snaps 266 may be part of the battery pack 204 and may be depressed into the battery pack 204 to release the pump housing 202 from the battery pack 204. When the snaps 266 are depressed into the battery pack 204, hooks 268 may be released from recesses 270 of the pump housing 202. In other embodiments, the snaps 266 may be manipulated in another way to release the battery pack 204 from the pump housing 202. In other embodiments, not pictured herein, the snaps 266 may be disposed within the pump housing 202 or the battery pack 204 such that a user cannot access the snaps 266. When disposed within the pump housing 202 or the battery pack 204 the snaps 266 may couple the pump housing 202 to the battery pack 204 but the pump housing 202 and the battery pack 204 may not be separated by a user.

In some embodiments, the battery pack 204 may further include an extension 272 that may extend into a center recess 274 of the pump housing 202 when the pump housing 202 and the battery pack 204 are coupled together. In some embodiments, the battery pack connection 258 may extend into the extension 272 and the housing connection 234 may extend into the center recess 274. When the pump housing 202 and the battery pack 204 are coupled, the housing connection 234 and the battery pack connection 258 may couple together to provide communication between the pump housing 202 and the battery pack 204.

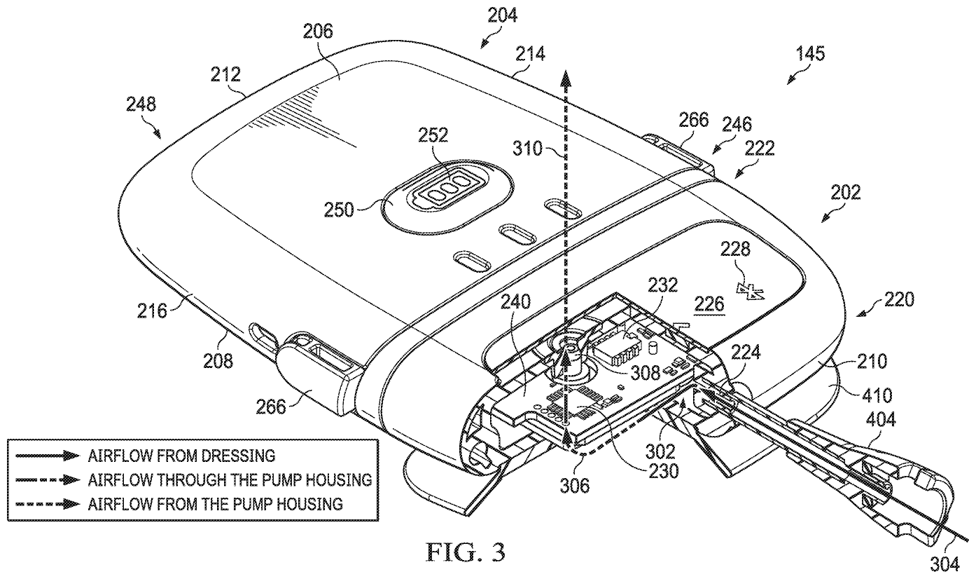

Referring to FIG. 3, the therapy device 145 is shown with the pump housing 202 including an optional sealed architecture. For example, the pump housing 202 may include an interior chamber 302 that may be hermetically sealed from an ambient environment. An inlet of the pump 230 may be directly exposed to the interior chamber 302 to generate negative pressure within the interior chamber 302 and an airflow 304 from the dressing 110 when the pump 230 is actuated. The airflow 304 may be drawn into the interior chamber 302 of the pump housing 202 and transition to an interior airflow 306 within the interior chamber 302. The interior airflow 306 may flow freely throughout the interior chamber 302 of the pump housing 202. The interior chamber 302 may include a one-way valve such as valve 308 that may enable the exhaust from an outlet of the pump 230 to be released from the interior chamber 302 of the pump housing 202. The valve 308 may couple to the port 229 such that the interior airflow 306 may travel from the inlet to the outlet of the pump 230 and through the valve 308 and escape from the pump housing 202 through the port 229. Once the interior airflow 306 has traveled through the port 229, it may become an exhaust airflow 310 that may combine with the ambient environment surrounding the therapy device 145.

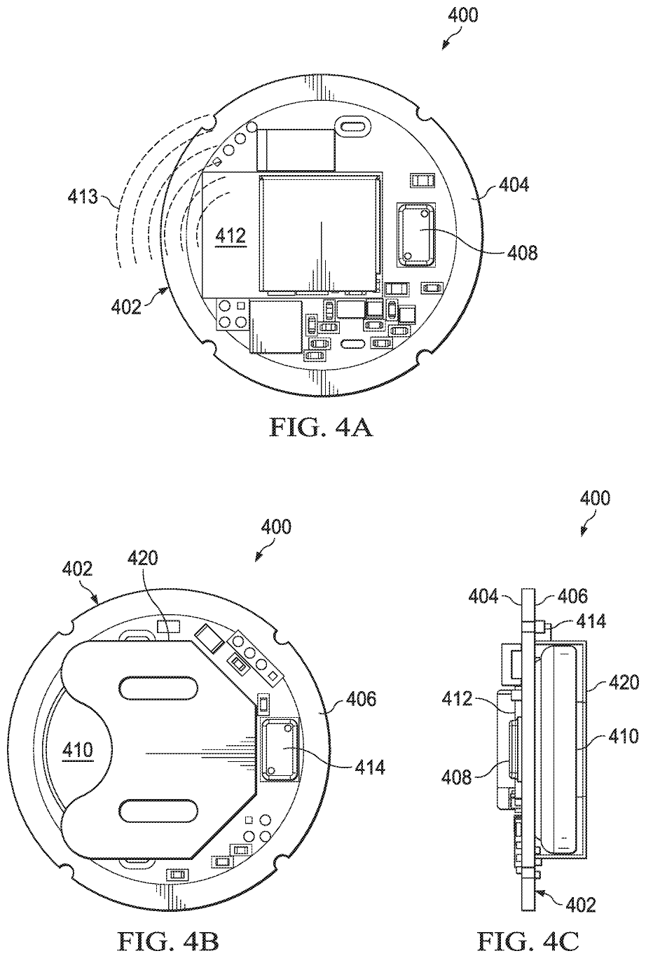

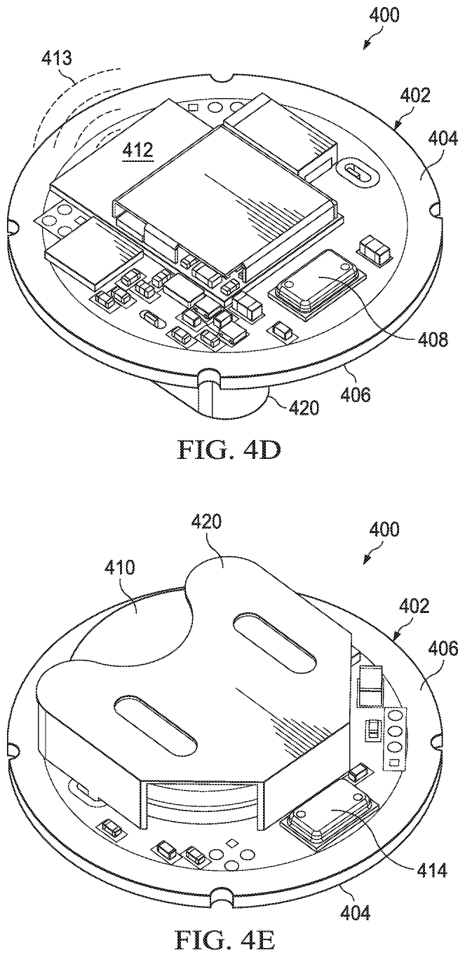

Referring collectively to FIGS. 4A-4E, these figures illustrate multiple views of an example embodiment of a pressure sensing device 400 that can be used with the therapy system 100. FIG. 4A is a top view of the pressure sensing device 400. FIG. 4B is a bottom view of the pressure sensing device 400. FIG. 4C is a side view of the pressure sensing device 400. FIG. 4D is a top perspective view of the pressure sensing device 400. FIG. 4E is a bottom perspective view of the pressure sensing device 400.

The pressure sensing device 400 may include a printed circuit board 402 with a first side or a first surface 404 and a second side or a second surface 406 opposite the first surface 404. The pressure sensing device 400 may further include a therapy pressure sensor 408, a battery or a power source 410, a second wireless communication device 412, and an ambient pressure sensor 414. In some embodiments, the therapy pressure sensor 408 may be coupled to the first surface 404 of the printed circuit board 402 and the ambient pressure sensor 414 may be coupled to the second surface 406 of the printed circuit board 402. Additionally, the power source 410 and the second wireless communication device 412 may be coupled to the first surface 404 of the printed circuit board 402. Alternatively, the power source 410 and/or the second wireless communication device 412 may be coupled to the second surface 406 of the printed circuit board 402.

When the pressure sensing device 400 is integrated into the therapy system 100, the therapy pressure sensor 408 may be configured to sense a pressure at a tissue site and the ambient pressure sensor 414 may be configured to sense an ambient environment pressure external to the therapy device 145 and the dressing 110 of the therapy system 100. In some embodiments the therapy pressure sensor 408 and the ambient pressure sensor 414 may create a single pressure sensing assembly that may be configured to sense a pressure at a tissue site and an ambient environment pressure.

The second wireless communication device 412 may be configured to wirelessly communicate with the therapy device 145 such that the components of the pressure sensing device 400 are communicatively coupled to the controller 130 of the therapy device 145. In some embodiments, the second wireless communication device 412 may be a Bluetooth® device. More specifically, the second wireless communication device 412 may be a Bluetooth® Low Energy system-on-chip that includes a microprocessor such as the nRF51822chip available from Nordic Semiconductor. The second wireless communication device 412 may be implemented with other wireless technologies suitable for use in a medical environment. The second wireless communication device 412 may be configured to wirelessly communicate a pressure signal 413 to the first wireless communication device 238 of the therapy device 145. In some embodiments, the pressure signal 413 may include a therapy pressure value corresponding to a pressure sensed at a tissue site by the therapy pressure sensor 408 and an ambient pressure value corresponding to an ambient environment pressure sensed by the ambient pressure sensor 414. When the therapy pressure sensor 408 and the ambient pressure sensor 414 are configured into the single pressure sensing assembly, the single pressure sensing assembly may be configured to generate the therapy pressure value relative to the ambient pressure value.

In some embodiments, the pressure sensing device 400 may further include a bracket 420 configured to secure the power source 410 in place on the pressure sensing device 400.

Referring to FIGS. 5A and 5B, an example embodiment of the therapy system 100 is shown. The therapy system 100 may include the therapy device 145 of FIGS. 2A-2F, the pressure sensing device 400, and the dressing 110.

The therapy device 145 may be coupled to the dressing 110 with a conduit 502. The conduit 502 may be a single lumen conduit that may couple the pump 230 of the therapy device 145 to the dressing 110. In some embodiments, the conduit 502 may have a first section 502a that may be coupled between the dressing connection 224 and a connector 504 and a second section 502b that may be coupled between the connector 504 and the dressing 110. The connector 504 may include a first half 506 and a second half 508. The first half 506 of the connector 504 may be coupled to the first section 502a of the conduit 502. In some embodiments, the first half 506 of the connector 504 may be directly coupled to the dressing connection 224 and the first section 502a of the conduit 502 may be omitted. The first half 506 of the connector 504 may be releasably coupled to the second half 508 of the connector 504. The first half 506 and the second half 508 of the connector may be coupled such that fluid may not escape from the therapy device or the conduit 502 through the connector 504. The second section 502b of the conduit 502 may extend from the second half 508 of the connector 504 to a dressing interface 510 that may be coupled to the dressing 110. The conduit 502 may be configured to distribute negative pressure from the pump 230 to the dressing 110.

The dressing interface 510 may have a dressing surface 514 that may be configured to couple to the cover 125 of the dressing 110. The dressing interface 510 may further include a conduit connection 516. The conduit 502 may be configured to couple to the dressing interface 510 at the conduit connection 516. The conduit connection 516 may be a first end 518 of a negative pressure path 520 that extends through the dressing interface 510. The negative pressure path 520 may have a second end 522 that extends through the dressing surface 514 of the dressing interface 510. The second end 522 of the negative pressure path 520 may couple with an opening 524 of the cover 125 such that negative pressure can be distributed through the dressing interface 510 to the dressing 110.

The pressure sensing device 400 may be disposed within the dressing interface 510 at the sensing location 503 in some embodiments. The pressure sensing device 400 may be disposed between the dressing surface 514 and an exterior surface 526 of the dressing interface 510. The dressing interface 510 may further include a circumferential edge 528 that may couple the dressing surface 514 to the exterior surface 526. The printed circuit board 402 may couple to the circumferential edge 528 of the dressing interface 510 such that the first surface 404 of the printed circuit board 402 is facing the dressing surface 514 of the dressing interface 510 and the second surface 406 of the printed circuit board 402 is facing the exterior surface 526 of the dressing interface 510. In some embodiments, the exterior surface 526 of the dressing interface 510 may include a vent 529 or an opening that may expose the ambient pressure sensor 414 to an ambient environment pressure external to the therapy system 100. The vent 529 may be isolated from the negative pressure path 520 such that pressure from the pump 230 is not able to leak or escape to ambient environment surrounding the therapy system 100.

The dressing interface 510 may further include a pressure sensing pathway 530 that may couple the therapy pressure sensor 408 to the dressing 110. More specifically, the pressure sensing pathway 530 may couple with a second opening 532 of the cover 125 of the dressing 110 such that the first surface 404 of the printed circuit board 402 is exposed to a pressure at the tissue site. The negative pressure path 520 may be isolated from the pressure sensing device 400 as well as the pressure sensing pathway 530.

In some embodiments, the dressing 110 may be an absorbent dressing that may be adapted to store fluid from a tissue site within the dressing 110. The dressing 110 may include the cover 125, the tissue interface 120, a film layer 534, and a release liner 536. The film layer 534 may be configured to contact the tissue site when the dressing 110 is disposed at the tissue site, the tissue interface 120 may be coupled to the film layer 534 opposite the tissue site, the cover 125 may be coupled to the tissue interface 120 opposite the film layer 534 and the release liner 536 may be removably coupled to the film layer 534 opposite the tissue interface 120 prior to the dressing 110 being placed at the tissue site.

Referring to FIG. 6, another example embodiment of the therapy system 100 is shown. The therapy system 100 of FIG. 6 may be similar to the therapy system of FIGS. 5A and 5B, but may include a container 602 coupled inline between the dressing 110 and the therapy device 145. The container 602 may be a fluid storage container that may be configured to collect fluids from the tissue site and prevent the fluids from reaching the pump 230 of the therapy device 145. The container 602 may be coupled inline between the pump 230 of the therapy device 145 and the dressing 110 through the conduit 502. The conduit may include the first section 502a coupled between the therapy device 145 and the connector 504, the second section 502b coupled between the connector 504 and a first side 604 of the container 602 and a third section 606 that may be coupled between a second side 608 of the container 602 and the dressing 110. Each of the first section 502a, the second section 502b, and the third section 606 of the conduit 502 may be a single lumen conduit.

Referring to FIGS. 7A and 7B, another example embodiment of the therapy system 100 is shown. The therapy system 100 may include the therapy device 145 of FIGS. 2A-2F, the pressure sensing device 400, and the dressing 110.

The pressure sensing device 400 may be carried by a conduit connection assembly 702 in some embodiments. The conduit connection assembly 702 may couple inline between the therapy device 145 and the dressing 110 at the sensing location 703. The conduit connection assembly 702 may include a first end 704 that may couple to the first half 506 of the connector 504 and a second end 706 that may couple to the second half 508 of the connector 504. More specifically, the conduit connection assembly 702 may include a first connector 708, a single lumen conduit 710, the pressure sensing device 400, a multi-lumen conduit 712, and a second connector 714. The first connector 708 may be configured to couple with the first half 506 of the connector 504 and the second connector 714 may be configured to couple with the second half 508 of the connector 504. The first section 502a of the conduit 502 may be a single lumen conduit that may couple to the first half 506 of the connector 504. The second section 502b of the conduit 502 may be a multi-lumen conduit and may couple to the second half 508 of the connector 504.

In some embodiments, the pressure sensing device 400 may be surrounded by a housing 716 that may have a first opening 718 to couple the housing 716 to the single lumen conduit 710 and a second opening 720 to couple the housing 716 to the multi-lumen conduit 712. The housing 716 may further include a first end 722 and a second end 724 opposite the first end 722. The first surface 404 of the printed circuit board 402 may be configured to face the second end 724 of the housing 716 and the second surface 406 of the printed circuit board 402 may be configured to face the first end 722 of the housing 716. The printed circuit board 402 may be configured to couple to the housing 716 such that the therapy pressure sensor 408 may be isolated from the ambient pressure sensor 414. The therapy pressure sensor 408 may be coupled to a sensing pathway 728 of the multi-lumen conduit 712. The sensing pathway 728 of the multi-lumen conduit 712 may be configured to fluidly couple the therapy pressure sensor 408 to the dressing 110 so that the therapy pressure sensor 408 may sense a pressure at the dressing 110. The sensing pathway 728 may extend from the therapy pressure sensor 408, through the multi-lumen conduit 712, and through the second section 502b of the conduit 502 to the dressing interface 510 and the dressing 110.

The multi-lumen conduit 712 may further include a reduced-pressure pathway 730 that may be isolated from the sensing pathway 728. The reduced-pressure pathway 730 may fluidly couple the pump 230 of the therapy device 145 to the dressing 110. The reduced-pressure pathway 730 may be fluidly coupled to the first section 502a of the conduit 502 and may extend through the second section 502b of the conduit 502 to the dressing interface 510 and the dressing 110. The sensing pathway 728 and the reduced-pressure pathway 730 of the multi-lumen conduit 712 and the second section 502b of the conduit 502 may extend through the dressing interface 510. The sensing pathway 728 may couple with a first opening 732 of the cover 125 and the reduced-pressure pathway 730 may couple with a second opening 734 of the cover 125. The first opening 732 may fluidly couple the sensing pathway 728 with the tissue interface 120 and the second opening 734 may fluidly couple the reduced-pressure pathway 730 with the tissue interface 120.

In some embodiments, the first end 722 of the housing 716 may include a vent 736 or an opening that may expose the ambient pressure sensor 414 to an ambient environment pressure external to the therapy system 100. The vent 736 may be isolated from the therapy pressure sensor 408, the sensing pathway 728, and the reduced-pressure pathway 730 such that pressure from the pump 230 is not able to leak or escape to ambient environment surrounding the therapy system 100.

Referring to FIGS. 8A-8C, another example embodiment of the therapy system 100 is shown. The therapy system 100 may include the therapy device 145, the conduit 502, the dressing interface 510, and the dressing 110. The therapy system 100 may further include a canister 802 that may be similar to the container 115 described above with reference to FIG. 1. The canister 802 may include a lid 804 and a body 806. The lid 804 may be coupled to the second surface 208 of the therapy device 145. The canister 802 may have a first end 808 and a second end 810 opposite the first end 808. The first end 808 of the canister 802 may be configured to couple with the conduit 502. The conduit 502 may be a multi-lumen conduit that may include a negative pressure lumen 814 and a sensing lumen 816. In some embodiments (not pictured herein), the conduit 502 may include a central lumen that may be the negative pressure lumen 814 that may be surrounded by a plurality of surrounding lumens. At least one of the surrounding lumens may be the sensing lumen 816.

The printed circuit board 402 may be disposed within the canister 802 at the sensing location 803 in some embodiments. The second surface 406 of the printed circuit board 402 may be facing the lid 804 of the canister 802 and the first surface 404 of the printed circuit board 402 may be facing away from the lid 804 of the canister 802. There may be a vent or an opening through the lid 804 or the body 806 in some embodiments that may expose the ambient pressure sensor 414 to an ambient environment surrounding the therapy device 145 and the canister 802. The vent or the opening may be configured such that it is isolated from any contents of the canister 802. In some embodiments, there may be a location tag 820 disposed within the canister 802 proximate to the second surface 406 of the printed circuit board 402. The location tag 820 may be an RFID tag in some embodiments. The location tag 820 may be configured to communicate with the therapy device 145 and may communicatively couple the pressure sensing device 400 to the therapy device 145 when the pressure sensing device 400 is within a predetermined distance or a sensing range of the therapy device 145.

The negative pressure lumen 814 may be configured to couple the pump 230 of the therapy device 145 to the dressing 110. More specifically, the negative pressure lumen 814 may couple to a pump connection on the second surface 208 of the therapy device 145. The pump connection may be disposed through the pump housing 202 and may fluidly couple the pump 230 to the dressing 110. The sensing lumen 816 may be configured to couple the therapy pressure sensor 408 to the dressing 110. The sensing lumen 816 may be configured to isolate the therapy pressure sensor 408 from the negative pressure lumen 814 and from an interior 822 of the canister 802 such that the therapy pressure sensor 408 is configured to sense a pressure at the dressing 110.

Also described herein is a method of treating a tissue site with negative pressure. In some example embodiments, the method can include obtaining the therapy device 145 including the pump 230, the pump pressure sensor 232, the first wireless communication device 238, and the controller 130. The pump 230 can be configured to deliver negative pressure, the pump pressure sensor 232 can be configured to sense a pressure at the pump 230, and the controller 130 can be configured to control the pump 230. The method can further include obtaining the pressure sensing device 400 including the printed circuit board 402, the therapy pressure sensor 408, the ambient pressure sensor 414, and the second wireless communication device 412. The therapy pressure sensor 408 can be configured to sense a pressure at the tissue site, the ambient pressure sensor 414 can be configured to sense an ambient environment pressure, and the second wireless communication device 412 can be configured to wirelessly communicate a pressure signal to the first wireless communication device 238. The pressure signal can include a therapy pressure value corresponding to the pressure at the tissue site and an ambient pressure value corresponding to the ambient environment pressure. The method can further include fluidly coupling the pump 230 to the dressing 110 disposed at the tissue site, fluidly coupling the pressure sensing device 400 to the dressing 110 disposed at the tissue site, actuating the pump 230 to deliver negative pressure to the dressing 110, and monitoring, with the pressure sensing device 400, the dressing 110 while the pump 230 is delivering negative pressure to the dressing 110.

In some example embodiments, the method can further include collecting fluids from the tissue site in a canister such as the container 602 or the canister 802. The canister can be configured to be coupled in fluid communication between the pump 230 and the dressing 110.

In some example embodiments, monitoring, with the pressure sensing device 400, the dressing 110 while the pump 230 is delivering negative pressure to the dressing 110 can include comparing the pressure signal and a pump pressure measured by the pump pressure sensor 232 to detect blockages within the dressing 110 or between the therapy device 145 and the dressing 110.

The systems, apparatuses, and methods described herein may provide significant advantages. For example, the therapy device 145 may provide a modular device that can be modified to use with different types of dressings and wounds. The therapy device 145 may be supplied in discrete parts or may be supplied as an assembled unit. The therapy device 145 can be modified as health care providers see fit and provides a solution that is in one product for case of use and flexibility. Further, the pressure sensing device 400 may be disposed at different locations depending on the therapy system 100 that is being used at a tissue site. The pressure sensing device may be disposed at the dressing 110 or between the therapy device 145 and the dressing 110 which may increase the accuracy of pressure measurements of the dressing 110 while also simplifying the pneumatic pathways of the therapy system 100 and reducing the cost and the chances of potential complications with the therapy system 100.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A system for treating a tissue site, the system comprising:
a dressing configured to be positioned at the tissue site;
a therapy device configured to fluidly couple to the dressing, the therapy device comprising:
a pump configured to deliver negative pressure to the dressing,
a first wireless communication device, and
a controller configured to control the pump; and
a pressure sensing device configured to be fluidly coupled to the dressing at a sensing location external to the therapy device and to wirelessly communicate a pressure signal from the sensing location to the first wireless communication device.

2. The system of claim 1, wherein the therapy device further comprises:
a pump pressure sensor configured to sense a pressure output of the pump; and
a power source configured to provide power to the therapy device.

3. The system of claim 1, wherein the therapy device further comprises:
a pump housing configured to contain the pump and including at least one housing connection configured to provide communication with the pump through the pump housing; and
a battery pack configured to couple to the pump housing, the battery pack including a power source configured to provide power to the therapy device and at least one battery pack connection configured to provide communication with the power source, wherein the at least one battery pack connection is configured to couple to the at least one housing connection when the battery pack is coupled to the pump housing.

4. (canceled)

5. (canceled)

6. The system of claim 1, wherein the pressure sensing device comprises:
a printed circuit board comprising a first side and a second side;
a therapy pressure sensor configured to sense a pressure at the tissue site;
an ambient pressure sensor configured to sense an ambient environment pressure external to the therapy device and the dressing; and
a second wireless communication device configured to wirelessly communicate a pressure signal to the first wireless communication device, wherein the pressure signal includes a therapy pressure value corresponding to the pressure at the tissue site and an ambient pressure value corresponding to the ambient environment pressure.

7. (canceled)

8. The system of claim 6, wherein the therapy pressure sensor is coupled to the first side of the printed circuit board, and wherein the ambient pressure sensor is coupled to the second side of the printed circuit board opposite the first side and isolated from the therapy pressure sensor.

9. The system of claim 8, wherein the pressure sensing device is coupled to a surface of the dressing opposite the tissue site, and wherein the therapy pressure sensor on the first side of the printed circuit board is configured to face the dressing and to be exposed to the pressure at the tissue site.

10. The system of claim 6, wherein the pressure sensing device is coupled to a surface of the dressing opposite the tissue site and configured to be exposed to the pressure at the tissue site.

11. The system of claim 10, wherein the therapy pressure sensor is fluidly coupled to the tissue site through the dressing.

12.-14. (canceled)

15. The system of claim 1, further comprising a conduit configured to fluidly couple the pump of the therapy device to the dressing.

16. The system of claim 15, wherein the conduit is a single lumen conduit extending from the pump to the dressing.

17. (canceled)

18. The system of claim 1, wherein the pressure sensing device is carried by a conduit connection assembly configured to be coupled between the therapy device and the dressing, the conduit connection assembly including a single lumen conduit connection at a first end and a multi-lumen conduit connection at a second end, the single lumen conduit connection configured to fluidly couple to a single lumen conduit between the pump and the first end, the multi-lumen conduit connection configured to fluidly couple to a multi-lumen conduit between the second end and the dressing.

19. The system of claim 18, wherein the multi-lumen conduit includes a reduced-pressure pathway fluidly isolated from a sensing pathway, wherein the sensing pathway is configured to be in fluid communication between the pressure sensing device and the dressing, and wherein the reduced-pressure pathway is configured to be in fluid communication between the dressing and the single lumen conduit fluidly coupled to the pump.

20. The system of claim 1, further comprising a fluid storage container configured to be coupled to an exterior of the therapy device and fluidly coupled between the pump and the dressing.

21. The system of claim 20, wherein the pressure sensing device is disposed within the fluid storage container.

22. The system claim 21, further comprising a negative pressure pathway configured to fluidly couple the pump to the dressing and a sensing pathway configured to fluidly couple the pressure sensing device to the dressing, wherein the pressure sensing device is not fluidly coupled to the therapy device.

23. The system of claim 22, wherein at least a portion of the negative pressure pathway and the sensing pathway are carried by a multi-lumen conduit configured to be fluidly coupled between the fluid storage container and the dressing.

24. The system of claim 22, wherein the sensing pathway is fluidly isolated from the negative pressure pathway.

25. The system of claim 1, wherein the pressure sensing device includes an RFID tag configured to communicatively couple the pressure sensing device to the therapy device when the RFID tag is within a sensing range of the therapy device.

26. A system for treating a tissue site, the system comprising:
a dressing configured to be positioned at the tissue site;
a therapy device comprising a pump and a wireless communication device, the pump configured to generate a negative pressure and to provide the negative pressure to the dressing through a negative pressure pathway; and
a pressure sensing device configured to be fluidly coupled to the dressing through a sensing pathway and at a sensing location external to the therapy device, the sensing pathway fluidly isolated from the negative pressure pathway and the therapy device, the pressure sensing device further configured to wirelessly communicate a pressure signal from the sensing location to the wireless communication device.

27. A method of treating a tissue site with negative pressure, the method comprising:
obtaining a therapy device comprising:
a pump configured to deliver negative pressure,
a pump pressure sensor configured to sense a pressure at the pump,
a first wireless communication device, and
a controller configured to control the pump;
obtaining a pressure sensing device comprising:
a printed circuit board,
a therapy pressure sensor configured to sense a pressure at the tissue site,
an ambient pressure sensor configured to sense an ambient environment pressure, and
a second wireless communication device configured to wirelessly communicate a pressure signal to the first wireless communication device, wherein the pressure signal includes a therapy pressure value corresponding to the pressure at the tissue site and an ambient pressure value corresponding to the ambient environment pressure;
fluidly coupling the pump to a dressing disposed at the tissue site;
fluidly coupling the pressure sensing device to the dressing disposed at the tissue site;
actuating the pump to deliver negative pressure to the dressing; and
monitoring, with the pressure sensing device, the dressing while the pump is delivering negative pressure to the dressing.

28. (canceled)

29. The method of claim 27, wherein monitoring, with the pressure sensing device, the dressing while the pump is delivering negative pressure to the dressing comprises comparing the pressure signal and a pump pressure measured by the pump pressure sensor to detect blockages within the dressing or between the therapy device and the dressing.

30. (canceled)
